(19) United States
(12) Patent Application Publication
(10) Pub. No.: US 2022/0115099 A1
(43) Pub. Date: Apr. 14, 2022

(54) ELECTRONIC HEALTH RECORD SYSTEM AND METHOD

(71) Applicant: Jurgen K. Vollrath, Sherwood, OR (US)

(72) Inventor: Jurgen K. Vollrath, Sherwood, OR (US)

(21) Appl. No.: 17/499,412

(22) Filed: Oct. 12, 2021

Related U.S. Application Data
(60) Provisional application No. 63/204,618, filed on Oct. 14, 2020.
(60) Provisional application No. 63/258,047, filed on Apr. 7, 2021.

(51) Int. Cl. G16H 10/60; G16H 40/67

(52) U.S. Cl. CPC G16H 10/60; G16H 40/67

(57) ABSTRACT

An EHR system and method that includes medical device data, voice transcription data and image data to corroborate data from multiple sources and aid in the allocation of data to fields in a user interface.

### FIELD OF THE INVENTION

The invention relates to Electronic Health Records and improved diagnosis and assessment of disease states.

### BACKGROUND OF THE INVENTION

A variety of electronic health records (EHRs) have been created, ostensibly to simplify record keeping for physicians and to provide for greater compliance and transparency of reimbursable procedures that are performed.

However, a common complaint from physicians is the difficulty of using these EHR systems. It is therefore not uncommon for physicians to resort to traditional means of jotting down by hand, a few notes regarding the procedures performed, their findings, diagnosis, and any prescriptions, and then communicating this to a nurse or physician's assistant to have an administrative staff member enter it into the system.

The many layers of communication and initial rudimentary notes by a clinician, are prone to error, and defeat the purpose of the electronic medical record system.

Even EHR systems that rely on voice transcription for data entry require laborious identification of the correct portal locations and checking of the transcripts in order to avoid any clinical data being entered in the incorrect location or being wrongly transcribed, e.g., data relating to procedures, patient medical condition, findings, diagnosis, prescriptions, referrals, follow-up, etc.

A further problem with current electronic health records (EHR systems) is that the data sources are usually very limited. These my include simply the clinical inputs entered by a clinician at the time of a clinician-patient interaction. Pre-existing data, such as clinical data obtained by a previous primary care physician for a patient, or by a specialist may not appear in a clinician's EHR system. Other data sources, such as lab and imaging data may also be found on separate systems.

The day-to-day changes in the physiological condition of a patient are also not reflected in current EHR systems, even though numerous data capture devices, like FitBits and other wearable devices, as well as non-wearable monitoring devices, such as radar fall detectors, are continually gathering data about people.

Even though ad hoc tools such as electronic databases and programs are available to determine the potential for adverse drug interactions prior to prescribing a new medication, these are not necessarily integrated into existing EHR systems and require diligence on the part of the clinician to verify such information separately.

### SUMMARY OF THE INVENTION

The present invention comprises a method and system that captures ground truth data both prior to and during a clinician-patient interaction. During a clinician-patient interaction data is captured using electronic sensors including medical, verbal, and visual sensors, which replace or at least limit the need for a clinician to write up or type in details of the interaction with the patient, thereby saving the clinician time that would otherwise be spent on follow-up administrative compliance tasks.

According to one aspect of the invention, there is provided an EHR system that includes a processor, control memory connected to the processor, data storage, at least one EHR user interface (UI), a microphone, a natural language processor (NLP), and at least one medical device that captures medical data about a patient and is in communication with the EHR UI, wherein the data from each medical device is associated with a defined location (also referred to herein as a field) in the EHR UI.

For purposes of this invention “communication with the UI” includes indirect communication, wherein the medical device communicates with a remote data storage. The processor may be a remote processor that controls the storing of medical data. Thus, the processor may be connected to a control memory that includes machine-readable code defining one or more algorithms for controlling the processor. For purposes of this application, the machine-readable code can be implemented as software logic or hardware logic. For example, in a hardware logic implementation, instead of having a separate processor and control memory with machine readable code, the processor and logic functions may be combined into a logical hardware unit, e.g. an application specific integrated circuit (ASIC) or field programmable gate array (FPGA). The EHR UI may comprise a portal defined by a web application (web app) accessible through a browser on a user device (using WiFi or a cell phone connection) or a native mobile application (App) that is downloaded to the user device.

The system may thus include a portal that defines one or more user interfaces (UIs).

In order to provide a wholistic view of a patient's condition data about a patient may include not only data captured in clinician-patient interactions but may include previously captured data.

Types of Data that may be included in the portal of the system, include:
1. Patient General information—demographics, family history, insurance, etc.
2. Pre-existing patient-specific clinical data, e.g., EHR data about a patient, ported from another system, lab data, imaging data, etc.
3. Continual or ongoing patient-specific data—e.g. wearables and non-wearables (e.g. camera and radar image capture). For purposes of this application continual means on an ongoing basis, which need not necessarily be continuous or regularly captured data capture but continues to add to the store of captured physiological and/or psychological information about a patient, e.g., wearables such as FitBit or monitoring devices such as fall-detectors in a home. Such information informs about changes over time and anomalies in the health of a patient. Ongoing data may also include data from the patient's interaction with others, e.g., social media data.
4. Ad hoc clinical data associated with clinician-patient interaction (in-office or remote), which was briefly discussed above, and may include:
   a. Physiological data capture using electronic medical devices. As is discussed further below, the different types of clinician-patient interaction data elements may be defined as Resources to facilitate the mapping of the data to specific data locations in a data structure and with defined locations or fields in a user portal)
   b. Verbal interactions (typically involving a microphone and which may include natural language processing (NLP) to transcribe and analyze the data). By semantically parsing the transcribed data, the parsed information supports correlation of verbal data by natural language processing (NLP) with a dictionary of terms (medical terminology, and instructional words), in order to:
      i. supplement electronic medical device data (e.g., instructions by the clinician to the patient, such as “Bend forward and breath in deeply” will help to identify what part of the patient body was being monitored, in order to associate medical device data with specific data locations in a data structure and with defined location in a user portal;
      ii. based on semantic parsing and a dictionary of medical terminology, in conjunction with voice transcription, it allows phrases to be identified as clinician supplementary notes for capture in Comment blocks.
   c. Video data (e.g., using one or more Wifi-enabled cameras) to:
      i. provide additional physiological and psychological data about the patient, e.g., pallor of the patient's skin, and emotional state of patient.
      ii. complement data from medical devices e.g., in order to define the location on the patient's body being monitored to assist in mapping medical device data to the correct data location.
      iii. provide a record of patient-clinician interaction for legal reasons, e.g., avoiding allegations of abuse or inappropriate behavior. Privacy of patients may be protected by generating avatars for the clinician and patient.
5. General symptomatic, genetic, and biologic lab data, correlated to diagnostic data, e.g., based on medical journal information, research, lab data, and third party EHR systems.

The system may include one or more of: a scheduling module, reporting module (also referred to herein as a reimbursement module), and patient billing module. The EHR UI may include multiple portals, including Clinician portal, Patient portal, Administrative portal, and Payer portal. The Clinician UI may define data fields that are dedicated to one or more of: procedures performed on a patient, patient medical condition information, medical findings, diagnosis, and prescriptions for the patient.

As mentioned above, the system may include at one or more image capture devices. These may comprise an RGB (visual spectrum: red, green, blue) video camera for monitoring the activities of a medical practitioner in relation to the patient (also referred to herein as a procedure image capture device or clinician-patient-interaction image capture device). Image data from the at least one procedure image capture device may be parsed for analysis and mapping to one or more data locations or fields.

As mentioned above, the control memory may include machine readable code configured to define one or more algorithms for controlling the processor to capture and map information from multiple data input sources. This may include parsing either the verbal (sound files) or voice-transcription data (e.g., using a combination of speech-to-text and optical character recognition.) For ease of reference these techniques will generally be referred to herein as natural language processing (NLP). Similarly, this may include mapping image data from the camera(s) to the UI where the data is to be displayed, and keeping a record of the data in the data storage.

Apart from the at least one procedure image capture device, one or more of the electronic medical devices may include their own image capture devices (e.g. RGB camera or infra-red (IR) camera) for capturing images of the regions of a patient that the medical practitioner is looking at.

The processor may further be controlled by an algorithm to corroborate data from multiple data input sources, and flag physiological discrepancies or anomalies.

The data input sources for clinician-patient interactions may include the microphone, the one or more medical devices, and the one or more procedure image capture devices. In order to edit or add to data patient data, the clinician UI may support user input sources, e.g., a keyboard (which may include a physical or electronic keyboard and/or keypad) and/or a touch-sensitive screen, wherein the machine-readable code may include logic to allow a clinician to drag and drop data into user-defined fields in the clinician UI.

The Patient UI may include only select data to assist a patient with scheduling, follow-up appointments, referrals, prescription fulfillment, and advisory health-care information.

The Payer UI may be limited to identifying the procedural steps performed and associated data in support of a reimbursement request.

The logic of the system associated with the reimbursement module, preferably tracks reimbursements and automatically calculates patient balance for processing by the patient billing module.

The referral module may include logic for searching and identifying referral sources (e.g., imaging, lab analysis, specialists, etc.) supported by a patient's insurance as defined by the patient general data.

Further, according to one aspect of the invention, there is provided an EHR system that includes a processor, control memory connected to the processor, data storage, a user interface (UI), at least one of: a procedure image capture device, and a microphone; and at least one medical device that captures medical data about a patient and is in communication with the UI, wherein the data from each medical device is associated with a defined location in the UI.

The procedure image capture device may comprise a video camera such as an RGB camera for capturing the activities and interactions between clinician and patient.

According to one aspect of the invention, there is provided a medical device that includes an image capture device, and communication means for transmitting image data captured by the image capture device, to a data storage. An example of such a medical device, includes an electronic otoscope with a camera, and Bluetooth or Wifi connection for transmitting electronic data to a data storage.

Further, according to the invention, there is provided a method of capturing patient information as part of a clinician-patient interaction (also referred to herein as a medical procedure or patient encounter), comprising providing a user interface (UI) with multiple data entry fields, providing one or more medical devices (also referred to herein as a medical instruments or medical sensors) that generate electronic data about physiological parameters of the patient, capturing the electronic data from each medical device and, displaying data from each device on the user interface in one or more data entry fields associate with said medical device, and capturing at least one of: voice data, and video data to supplement the electronic data from the medical devices.

The method may further include providing voice-transcription software to convert the voice data into text and entering the voice data into at least one data entry field in the UI. The method may further include parsing the video data, time stamping the video data, and correlating the electronic data captured from at least one of the medical devices with the video data for the corresponding time frame.

One or more of the medical devices may include an image capture device.

Data from the voice-transcription software may be associated with a data entry field based on one or more of key words and key phrases in the voice data, and the context of the key words and key phrases.

The voice data may be time stamped and parsed to identify key words and key phrase, and, in the case of a medical device being used during a corresponding time that the voice data is captured or within a defined time period of the voice data being captured, a key word or key phrase of the voice data may be used to provide additional information about the procedure that was performed using the medical device, e.g. the location on a patient's body that a stethoscope was applied to.

Insofar as voice data is associated with activities involving a particular electronic medical device, the voice data may be entered into the same or a related field as that of the medical device. Similarly, image data captured by the procedure image capture device during a time frame that a medical device is use, and image data captured by an image capture device associated with a medical device, may be displayed in a common or related field with medical data from said medical device.

The voice data, image data, and medical device data associated with a common or related field may also be used to corroborate each other. Any discrepancies between data from different data sources in the same or related field, may be flagged. Similarly, voice data, image data, and medical device data may be compared to pre-stored data in order to identify anomalies or physiological problems that should be flagged.

### BRIEF DESCRIPTION OF THE DRAWINGS

FIG. 1 a schematic representation of one embodiment of a system of the invention;

FIG. 2 shows one embodiment of an EHR user interface, and

FIG. 3 shows one embodiment of the logic involved in identifying the type of data received from a medical sensor and allocating it to the correct field in an EHR UI.

### DETAILED DESCRIPTION OF THE INVENTION

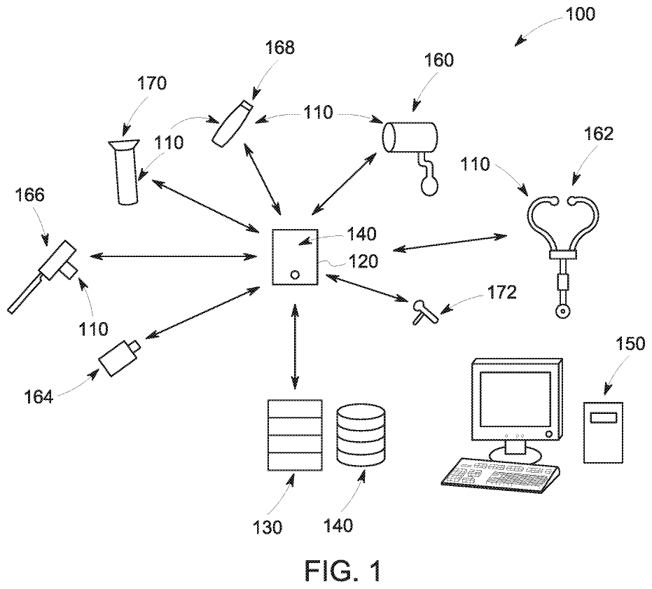

One embodiment of a system 100 of the invention is shown in FIG. 1. A doctor's office, hospital, or at-home patient monitoring system in this embodiment, includes multiple medical devices 110 that are connected, by short range communication, e.g., Bluetooth, or through wire connection to a hub 120.

In this embodiment the hub is defined by a smart phone that communicates by cell phone or Wifi connection with a remote server system 130, e.g., dedicated server, cloud server like Amazon Web Services (AWS) or edge server system. In another embodiment, instead of using a smart phone or separate hub to collect data from the medical devices, a laptop may be provided with a wireless receiver that plugs into the laptop's USB port for communicating by short range communication (e.g., Bluetooth) with the medical devices (as is done with Firefly's otoscope, which is discussed further below).

In this embodiment the smart phone 120 also provides a user interface (UI), for the user (typically a medical practitioner (also referred to herein as a clinician), e.g. physician, nurse, paramedic, etc) to view an electronic medical record (EMR) or electronic health record (EHR), which may be implemented as a web application (web app) accessible through a browser on the smart phone 120 (using WiFi or a cell phone connection) or a native mobile application (App) that is downloaded to the smart phone. For ease of reference the term EHR will be used in this application to refer to an EHR or EMR system. In the present embodiment, the EHR is provided on the server 130, and the smart phone 120 accesses the EHR as a web app on the server 130.

The medical devices 110 communicate via the hub (in this case, defined by the smart phone 120) with the server 130, which includes a control memory as part of the server, and a database 140 for storing patient data from the medical devices 110, as well as pre-stored data for comparing the patient data, as discussed further below.

The server 130 also provides a portal for users to access patient data. As is discussed in greater detail below, users may include clinicians, patients, payers, administrative staff, etc., each of which may be provided with a separate user interface to the portal, with access to such patient data as is appropriate for their needs. User access devices, such as a desktop 150 at a doctor's office are provided with communication access with the server 130, e.g., via the Internet, using a WiFi connection in order to access the portal of the EHR.

In this embodiment the medical devices that capture patient clinical data include a blood pressure cuff 160, e.g., the QardioArm by Qardio, which measures heart rate, systolic and diastolic blood pressures. In a traditional implementation of the QardioArm data is emailed to the user's physician. However, the present invention, the data is integrated into the EHR system of the invention. The smart phone 120 captures the patient data and either streams the data by WiFi to a memory storage such as the database 140, or has the smart phone 120 act as an edge processor to parse the data before sending it to the database 140, or process the parsed data by comparing it to pre-stored data in a data memory. Once the data has been parsed and analyzed for flagging conditions (also referred to herein as medical anomalies that may require further analysis). The source of the data (in this case a Qardio blood pressure cuff) provides context to map the data to the appropriate field in the EHR database 140 and user interface. This allows is the captured data to be used as a data input to automatically populate the correct field in the user interface, as is discussed on greater detail below.

The medical devices in this embodiment, further include an electronic stethoscope 162, such as the Eko device by Eko Solutions, which provides for stethoscope audio and ECG live streaming, including heart and lung sounds, identification of Atrial Fibrillation, heart murmurs, tachycardia, and bradycardia to assist providers in the detection and monitoring of heart disease. It also includes an integrated telemedicine platform for video conferencing with a medical practitioner. In accordance with the present invention, the medical data captured by the stethoscope is transmitted to the server 130 for processing and automatic population of the fields allocated to the stethoscope in a physician's user interface, which forms part of a portal of the EHR.

Another device included in the present embodiment, is a video camera 164. The camera 164 captures the activities of the medical practitioners as they are diagnosing or performing other medical activities on a patient. In this case the video camera is an RGB camera.

As with the medical devices 110, the data captured by the camera 164 is transmitted to the server 130 (in this case by Bluetooth to the hub 120 and using WiFi from the hub to the server 130).

The camera 164 is also referred to herein as a procedure image capture device since it captures the activities performed by the medical practitioner(s) as part of the medical procedure. In addition to the camera 164, some of the medical devices, such asotoscope 166 includes its own camera and electronic connection for wirelessly transmitting the data from the otoscope. One example of such a device is the Firefly by Firefly, which allows still images to be captured as image files, e.g., jpg, bmp, or video to be captured as video files, e.g., mov, avi. Firefly traditionally allows images and video clips to be uploaded manually into a physician's EHR. However, according to the present embodiment of the invention, the data from each of the medical devices 110 is associated with a unique device identifier in the data base. This allows clinical patient data from each of the devices 110 to automatically be mapped to fields in the clinician' sEHR system by associating the fields in the portal with field identifiers that are related to the device identifiers.

Similar to the otoscope 166, the present embodiment also includes additional medical devices 110, including a dermascope 168 with built-in camera for viewing the skin for lesions or growths; and an iris scope 170 with camera for viewing and evaluating the eyes of a patient. Example devices of the dermascope 168 and iris scope 170 are also provided by Firefly and, similar to the otoscope 166 described above, allow images to be transmitted electronically.

In this embodiment one further device includes a microphone 172 for capturing voice data from the interaction between the patient and the medical practitioner. This is transcribed into text and analyzed using natural language processing (NLP) software and having both an audio file and a text file of the interaction captured in the database 140. The NLP software may be provided on the server 130 or on an intermediate device such as the smart phone 120. In one embodiment, the NLP software analyzes the data from the microphone in order to identify keywords and key phrases and generates a text message for entering into the EHR system. By time-stamping the audio data and associating it with a medical device used in the same time frame or closely-related time frame (e.g. within 30 seconds of a key word or phrase in the audio data) additional information about the data from the medical device may be obtained to supplement the medical device data or help to appropriately allocate the audio data to the correct field in the EHR UI. Thus, the microphone 172 data serves multiple functions, including commentary by the clinician, which may be entered into one or more text fields in the portal, depending on the context (wherein the context may be derived from semantic parsing of the text file obtained from the audio data, as well as from the nature of other medical devices that are being used by the clinician at the time of the verbal input). The verbal inputs from the clinician and/or patient may also provide additional data for mapping information, e.g., by distinguishing different body parts being analyzed by the clinician, such as distinguishing left and right eye data captured by the iris scope 170, and thereby ensure that images of the left and right eye are allocated to the correct location in the physician's user interface of the EHR. In the present invention, data from the camera may similarly assist in providing additional data mapping information. Thus, data from the camera and microphone complement each other, where one source is unavailable or obscured. For instance, the physician may at times obscure the camera, in which case verbal input may supplement the missing image data. Similarly, if the physician fails to verbalize what he or she is doing, the camera may supplement this with visual data to assist in mapping medical device data.

As discussed above, the medical devices 110 may be connected wirelessly to a hub (as in the above embodiment) or by wire connection, e.g., to a modem for access to the Internet. While Bluetooth was used in the above embodiment, it will be appreciated that other connections could be implemented as are known in the art, e.g., wired connections such as Ethernet, USB, CAN, RS-232, RS-485, HDML, SATA, etc. or wireless connections such as direct to WiFi using an integrated modem, or via Bluetooth/BLE, 802.15.4/ZigBee, or GSM/CPRS, or using a custom/proprietary protocol.

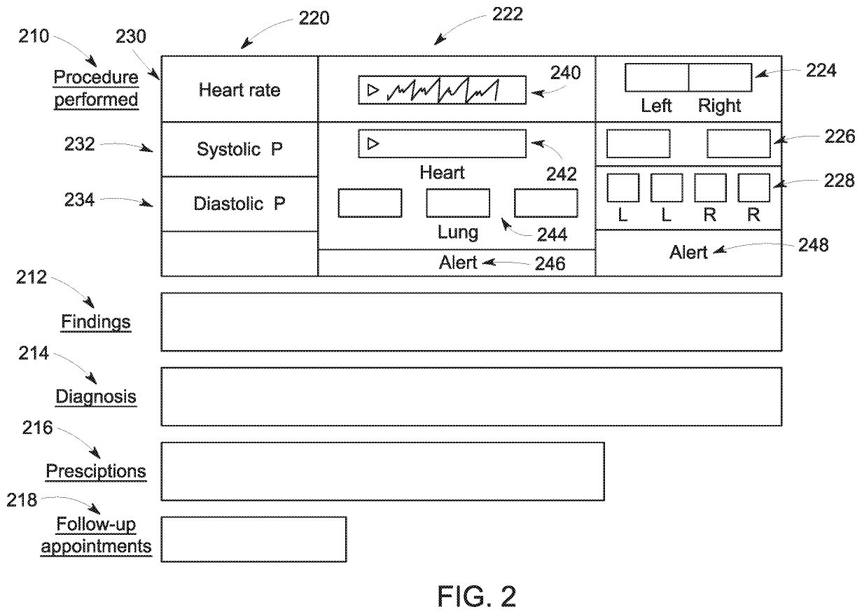

As indicated above, in order to populate the EHR system of the present invention, the one or more user interfaces of the portal associated with the EHR are divided into fields, each identified by a field identifier, and can include sub-fields, each with their own unique identifier. One such embodiment is shown in FIG. 2, and includes the fields:
“Procedures Performed” 210: which in this case includes sub-fields for measurements taken by the blood pressure cuff (fields 220); by the stethoscope (fields 222); by the otoscope (fields 224); by the dermascope (fields 226), and by the iris scope (fields 228), and may each include a free-text data entry field for adding parsed microphone data e.g., “listened to heart and lungs”, “viewed patient's ears with otoscope”, or “took pulse and blood pressure readings”,
“Findings” by the physician (field 212), e.g., “Patient's left ear canal was red in color with yellow discharge”,
“Diagnosis” (field 214)”, e.g., inner ear infection,
“Prescription” (field 216), e.g., antibiotics and painkillers of a defined type, and
“Follow-up appointments” with scheduling calendar 218.

The Findings, Diagnosis, and Prescription fields may each be populated from transcribed microphone data, wherein the correct mapping of the transcribed text data, to the appropriate field is derived from one or more of: semantic parsing of the text data, and the context of the data, e.g., as derived from a particular medical device 110 being used at the time of the verbal input, or derived from the image data captured by the camera 164 at the time of the verbal input. Thus, the camera and medical devices 110 assist in the mapping of transcribed text data, and similarly the data from the camera and microphone assist in mapping the data from the medical devices 110.

Data collected from the medical devices 110, camera 164, and microphone 172, may include text files, image files, video files, audio files, each defining an aspect of the encounter between the patient and the medical practitioner.

In this embodiment, the Procedures Performed field is divided into sub-fields, that correspond to defined medical devices 110. Thus, as mentioned above, the Procedures Performed field includes a sub-field 220 for data from the pressure cuff 160, a sub-field 222 for the stethoscope 162, a sub-field 224 for the otoscope 166 (left and right ear), a sub-field 226 for the dermascope 168 (for showing pictures of various skin conditions), and a sub-field 228 for the iris scope 170 (for showing pictures of each eye under different conditions).

The readings for the pressure cuff 160 and stethoscope 162 are further sub-classified into sub-fields. In the case of the pressure cuff 160, it includes sub-fields for heart rate (field 230), systolic blood pressure (field 232) and diastolic blood pressure (field 234). In the case of the electronic stethoscope 162, the sub-fields include a field for the ECG graph (field 240), audio files for heart sounds (field 242), audio files for lung sounds (fields 244) taken at various locations of the patient's body. It also includes artificial intelligence (AI) diagnostic information (field 246) in the form of an Alert or flag when an anomaly or aberration is detected in the audio or image data compared to a database of normal ECG profiles and normal heart and lung sounds, to identify problems such as Atrial Fibrillation, heart murmurs, tachycardia, and bradycardia.

As shown in FIG. 2, the iris scope data includes fields 228 for capturing images of the left and right eyes. It is also associated with an alert field 248 where an analysis of the eye images is compared to a database of images in database 140, which includes pre-stored images of health conditions (including eye problems, as well as systemic problems detectable from patients' eyes). While image data from the otoscope (field 224) and dermascope (field 226) in this embodiment, don't include an alert field, it will be appreciated that image data from these two medical devices can similarly be compared to images in the database 140 of medical problem conditions associated with ears and skin respectively, in order to generate alert messages.

The AI system, in one embodiment, may be implemented at the server by providing machine readable code on a control memory connected to a processor. The machine-readable code defines an algorithm for controlling the processor to parse incoming audio data from the stethoscope and compare this to pre-stored audio files in the database 140. Aberrations or anomalies in the audio data, indicative of one or more medical conditions, e.g. atrial fibrillation, are flagged (to define a flagging event) in the diagnostic information field of the EHR UI and a corresponding message is generated to populate the diagnostic field.

Image data from the video camera 164, and audio data from the microphone 172 are similarly parsed to identify and extract information to supplement data provided by the other medical devices 110 and also to assist in identifying the appropriate sub-fields in the EHR in cases where there is more than one field associated with a medical device. For instance, the stethoscope, may be used by a physician to listen to various regions of the patient's body to pick up different sounds, such as heart beat or breathing aberrations.

One example where the microphone data may supplement data from a medical device would be in the verbal interactions between a physician and a patient, which may indicate that the patient has a middle-ear infection in the left ear. The parsing of the audio data allows predefined terms or phrases such as “middle-ear” “ear”, “infection” to be identified from a dictionary of terms and phrases stored in the database 140, and the word “left” could be used to identify which ear is associated with an image of an inflamed ear.

In one embodiment, the terms and phrases in the dictionary may each be associated with a field in the EHR. An algorithm in the memory uses this information to identify the appropriate field to allocate the information to and to generate a message that is then added to the diagnostic

The algorithm, may be implemented as an AI system that gathers data from all of the medical devices 110, camera, and microphone, to identify overlaps or correlations in evidence from multiple medical device sources. Thus, as indicated above, the information from the microphone or video camera may also serve to corroborate the information gleaned from the data of a medical device 110. In this example, the image data from the otoscope 166 may be corroborated by the verbal data from the physician that the patient has a middle ear infection of the left ear, and the camera 164 may verify that the physician did in fact check the left ear of the patient, supplementing the otoscope data.

In order to correctly allocate the data to the relevant fields, one approach discussed above involves parsing data, such as audio and image data, and finding corresponding data amongst the pre-stored files in the database 140, which are each associated with a field or sub-field in the portal of the EHR, e.g., by means of field identifiers associated with specific pre-stored files in the database. Thus, the audio data from the microphone 17:2 and video data from the camera 164 may be parsed to permit comparison of the parsed data to pre-stored audio and image files, which are associated by means of field identifiers with specific fields in the portal of the EHR. Thus, by identifying correlations to the pre-stored data, the parsed data can be allocated to the appropriate EHR field.

Also, as discussed above, some of the medical devices 110 can be associated with specific fields, so that data from each of these devices is automatically associated with one or more fields in the UI.

In the case of a traditional doctor's visit at a physician's office, the start and end of a session may be defined by manually starting and ending the recording of the video camera and microphone. Data from the video camera or microphone is correlated with that of a medical device by time stamping the video and audio data, and relating it to time-stamped medical device data.

In another embodiment, one or more of the video camera and microphone are always on, but initially collecting data purely for purposes of making a determination whether to start monitoring a patient session (i.e., capturing and analyzing the data for purposes of assigning it to the appropriate fields in the EHR). This determination is based on start indictors (audio or visual cues) e.g. in the case of the microphone, listening for phrases or key words, such as: “Patient session start”, or “Please confirm your name and date of birth” or in the case of the camera, identifying when a patient and a medical practitioner (e.g., nurse or physician's assistant or physician) are both present in the room. The data from the camera and microphone may be continuously streamed and remotely processed by a processor, or the camera and microphone data may be locally processed, wherein a local processor identifies the start of a session. In this latter situation, the local processor may include a memory, which in one embodiment includes data memory configured with pre-defined phrases that define the start of a session, a processor, and control memory that is configured with machine readable code defining an algorithm that parses the data received from the video camera (e.g. using X-NECT or V-NECT software) and/or from the microphone (using NLP software). The end of a session is similarly determined by visual or audio cues, e.g., the video showing the patient leaving, or the medical practitioner making a verbal comment, e.g., “Patient session end”, “Have a good day”, “We will let you know when the results are in.”, “You can make an appointment for your follow-up at the front desk.”, etc.

In the case of a remote medicine (telemedicine session), the start and end of a session may be defined by the commencement and termination of a video call between the patient and medical practitioner, which may be initiated by either party.

As discussed above, in order to correlate data being captured by a medical device 110 (also referred to herein as a medical monitor or medical sensor) with that from the camera and/or the microphone, audio and video data packets from the microphone and video camera may be time stamped to relate them to corresponding time frames for the medical device. In one embodiment video data from the video camera, and audio data from the microphone are continuously streamed to a server system, e.g., via WiFi. A clock associated with a processor, which forms part of the server system, associates time information with the audio and video data. When a medical sensor, e.g., electronic stethoscope starts monitoring a patient and relaying data about heart rate or breathing, a time stamp is associated with the data. Similar to the microphone and video camera discussed above, the commencement of monitoring by a medical device, may be defined by switching on the medical device, or may be determined by logic on a control memory at the server location (or local or edge processor) listening for the sound of a heart beating or the sound of breathing and comparing this data to pre-stored data to identify that the sound received is in fact a beating heart or the sound of breathing, and thus the commencement of the medical device being used.

In another embodiment, or by way of corroboration, data from the video camera may be used to validate that the medical device (in this case the stethoscope) is being applied to the patient, and thus commence and time stamp the data captured by the stethoscope. As mentioned above, since the stethoscope may be applied to different regions of the patient's body, the video camera, also identifies where the stethoscope reading is being taken. As discussed, this identification of the body part being monitored may include parsing the video data and comparing it to pre-stored data in a data memory, e.g., database 140, which includes pre-stored visual data of different body locations of a patient. This allows anomalies detected by the stethoscope to be transferred to the correct field in the UI of the EHR as an alert or warning signal.

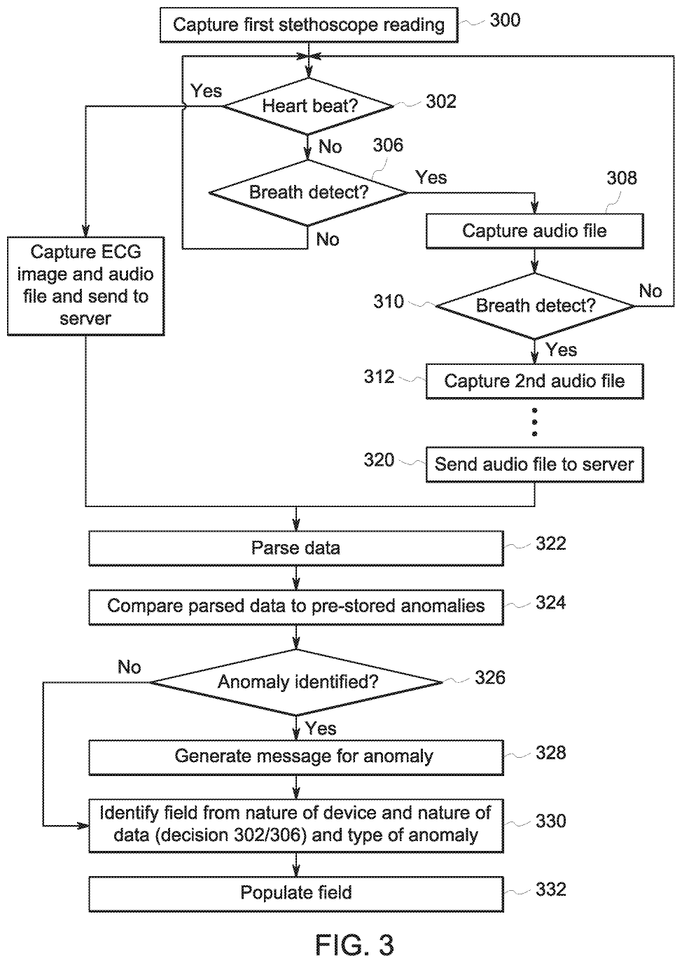

One embodiment of the logic for an algorithm to analyze data from the medical devices 110 and allocate them to the appropriate fields in the UI of the EHR, is shown in FIG. 3 with respect to data from the stethoscope 162.

Data is captured from the stethoscope 162 during a first reading taken by the stethoscope (block 300). Since the stethoscope can be used to detect breathing patterns and heart beat in this embodiment, decision block 302 determines whether the sound of a beating heart is detected. If yes, the data generated by the stethoscope, which includes both an audio file and an image file of the ECG pattern, is sent to the server (block 304).

If no heart sound is detected, decision block 306 makes a determination whether the sound of breathing is detected. If not, it loops back to take another reading. If breathing is detected, a first audio file is captured associated with a first location of the stethoscope on the patient (block 308), until breathing is no longer detected. Since the stethoscope may take readings at multiple locations, the algorithm again checks for breathing sound (decision block 310) and if breathing is again detected, a second audio file is captured (block 312). This is repeated up to block 320 until no breathing is detected. If no breathing sound is detected the logic loops back to determine whether either a heart beat or breathing is detected. If after a predefined number of attempts (loops) no sound is detected the audio files are sent to the server. It will be appreciated that after each data file is captured it can be sent to the server, or data from the stethoscope can be continuously streamed to the server 140 for analysis to identify the start and end of a measurement, the nature of the data, and the time stamp associated with the measurement.

In block 322 the logic parses the data and then in block 324 compares the parsed data to pre-stored anomaly data of heart beat anomalies and breathing anomalies. If a correlation to anomaly data is detected in decision block 326, the algorithm generates a corresponding pre-defined message associated with the identified anomaly (block 328). The logic then identifies a field in the portal of the EHR, which to populate with data (block 330), which in this case is based on the nature of the medical device 110 (stethoscope 162), the nature of the data (heart beat or breathing as defined by decision blocks 302, 306), and the type of anomaly as identified by decision block 326. With this information, the data is submitted by the processor to the EHR for entry into the defined field.

In order to implement the system of the invention, one embodiment includes a Scheduling Module, a Reporting Module (also referred to herein as a Reimbursement Module), and a Patient Billing module.

The Scheduling Module may define a separate Administrative User Interface (Administrative UI) dedicated to scheduling of appointments, and may include only certain patient information, such as Patient General Information. This may include patient name, contact information, demographics, family history, and patient insurance information. The Scheduling Module may also include the task of referring the patient to third party resources.

Alternatively, in one embodiment, this is performed by a separate Referral Module. This module serves to streamline the process of referring patients to specialists, or to have lab work or radiographic work done, etc. The Referral Module may include logic for searching and identifying referral sources (e.g., imaging, lab analysis, specialists, etc.) supported by a patient's insurance as defined by the patient general data, and may send out referral requests automatically. Alternatively, in one embodiment, the Referral Module may make the possible referral sources available to the patient in the Patient UI with a request that the Patient elect one or more of the sources in order of preference. Thus, the system may include additional data about each source: e.g., geographic location, and if applicable, specific physicians and experience levels, to allow the patient to make an informed decision. Since referral requests often have to be made by the referring physician, the patient's election may be linked back to the Scheduling module to have an administrative staff member formalize the referral.

The Reporting Module is directed to reimbursements, and may be associated with a Payer UI that includes only the patient information needed for a payer to confirm the procedures performed by a clinician in order to verify the CPT (current procedural terminology) code and request for reimbursement.

The Reporting Module may be implemented by an algorithm defined by machine-readable logic in the control memory of the server 130. This may include an algorithm that defines an Evaluation and Management (E/M) coding assistant to generate the OM codes for physician-patient encounters associated with the activities identified for the Procedures Performed field 210. These are then translated into CPT (current procedural terminology) codes to facilitate reimbursement by the patient's insurance. In one embodiment, the request for reimbursement is then automatically submitted to the payer.

The logic associated with the Reporting (Reimbursement module), preferably also tracks reimbursements and may calculate patient balance for processing and invoicing by the patient Billing Module.

In one embodiment the Patient Billing Module may perform both the function of calculating costs for which the patient is responsible, e.g., deductibles, and non-reimbursed costs, based on the patient's insurance information, as well as invoicing.

As indicated above, the portal may support multiple user interfaces (UIs) dedicated to the needs and authorizations of different users. Thus, the EHR system of the invention, may include data gathered from multiple sources, and presented to the various users by user-specific interfaces, wherein the data available to each UI depends on the user's access requirements. For example, an administrative person tasked with scheduling appointments may only have access to Patient General Information, whereas a physician may have access to all of the patient data, as defined below.

The main UI of the EHR system of the present invention is the Clinician UI, which, as discussed in detail above, captures pre-existing data about a patient from other EHR systems, and other clinical data systems via; captures patient clinical data in clinician-patient interactions; captures continual monitoring data of the patient, and captures third party data, such as research data.

In one embodiment, the portal also includes a Patient UI to allow a patient to access his or her clinical data, diagnosis, and follow-up information (referrals to specialists, lab work, prescriptions, follow-up appointments, etc). Thus, the patient UI may be linked to both a Scheduling Module (which records the patient's next schedule appointment), and to a Patient Billing Module (that calculates the patient's charges and invoicing information.) The Patient UI may include only select data to assist a patient with scheduling, follow-up appointments, referrals, prescription fulfillment, and advisory health-care information.

As mentioned above, the portal may also include a Payer UI, which may be limited to the procedural steps and associated data in support of a reimbursement request. Also, as discussed above, the portal may include an Administrative UI for administrative tasks like appointment scheduling.

According to one aspect of the invention, the system and method of the invention seeks to capture data from multiple sources, integrate it, and make it available on one platform that is accessible by different users according to their needs and authorization levels, taking into account patient privacy considerations. In particular, the data includes both patient-specific data and general medical data.

Traditionally, research has focused on the investigation of disease states based on the changes in physiology in the form of a confined view of a singular modality of data. This fails to recognize the variation and interconnectedness of the underlying medical mechanisms.

New technologies, however, make it possible to capture vast amounts of information about each individual patient. The present invention seeks to expand the parameters taken into account in diagnosing a patient and making care recommendations, by gathering a much broader range of data. This includes patient data gathered over an extended timescale by means of wearables and ambient sensors. It also includes the use of medical electronics coupled with corroborating and supporting data from cameras and microphones to speed up and improve the accuracy of patient clinical data capture within the realm of clinician-patient interactions.

Thirdly, the present invention seeks to gather data from third party sources—not only those relating to the patient, e.g., imaging and genomic data of the patient, but also imaging data and genomic data as it pertains to third parties and identified disease states.

Physiological and pathophysiological phenomena manifest as changes across multiple clinical streams due to strong coupling among different systems within the body (e.g., interactions between heart rate, respiration, and blood pressure) thereby producing potential markers for clinical assessment. Thus, understanding and predicting diseases requires an aggregated approach, taking into account the broad range of data sources mentioned above, where structured and unstructured data stemming from a myriad of clinical and nonclinical modalities are utilized for a more comprehensive perspective of the disease states.

The present invention thereby seeks to provide a more comprehensive overview of a patient's health and render a diagnosis of the patient's condition by capturing both patient-specific parameters and medical third-party data. As indicated above, the patient-specific parameters may be categorized into (a) long-term or continually-captured data (e.g., through wearables, ambient sensors such as radar fall detectors, and patient interactions with social media), and (b) clinician-patient interactions (which include discussions between clinician and patient, and monitoring of the patient's physiological parameters using electronic medical devices. This is supplemented with third-party data such as medical research, imaging data and genomic data.

Image Processing. Medical images are an important source of data frequently used for diagnosis, therapy assessment and planning For purposes of this application, the term image data includes Computed tomography (CT), magnetic resonance imaging (MRI), X-ray, molecular imaging, ultrasound, photoacoustic imaging, fluoroscopy, positron emission tomography-computed tomography (PET-CT), and mammography are some of the examples of imaging techniques that are well established within clinical settings. Medical image data can range anywhere from a few megabytes for a single study (e.g., histology images) to hundreds of megabytes per study (e.g., thin-slice CT studies comprising up to 2500+ scans per study). In addition, other sources of data acquired for each patient may be utilized during the diagnoses, prognosis, and treatment processes.

Medical device Signal Processing. Data from electronic medical devices pose a challenge from a spatiotemporal nature. Analysis of physiological signals is often more meaningful when presented along with situational context awareness which needs to be embedded into the development of short-term and continual monitoring and predictive systems to ensure its effectiveness and robustness and avoid alarm fatigue due to flagging of false positives.

Traditional approaches have failed primarily because they tend to rely on single sources of information while lacking context of the patients' true physiological conditions from a broader and more comprehensive viewpoint. Therefore, there is a need to develop improved and more comprehensive approaches towards studying interactions and correlations among multimodal clinical time series data.

Genomics. One approach that has been proposed for integrating genomic data into predictions is the predictive, preventive, participatory, and personalized health, approach referred to as P4. It uses a system approach for (i) analyzing genome-scale datasets to determine disease states, (ii) moving towards blood based diagnostic tools for continuous monitoring of a subject, (iii) exploring new approaches to drug target discovery, developing tools to deal with big data challenges of capturing, validating, storing, mining, integrating, and (iv) modeling data for each individual, with the hope of ultimately, realizing actionable recommendations at the clinical level remains. The present invention seeks to adopt genomics as one of its data sources—both from the patient's genomic data, and as a source of relating third party genomic data to identified disease states.

Thus, one embodiment of a system of the invention may include multiple data sources that are integrated onto a single platform. These may include:
1. Patient General information—(patient name, contact information, demographics, family history, insurance, etc.)
2. Pre-existing patient-specific clinical data, e.g., EHR data about a patient, ported from another system, lab data, imaging data, etc.
3. Ongoing patient-specific data (data captured about a patient during day-to-day activities)—e.g., wearables (such as FitBit) and non-wearables (e.g., information captured by a camera or radar image detector mounted in the home of the patient). For purposes of this application continual means on an ongoing basis, which need not necessarily be continuous or at regular intervals but continues to add to the store of captured physiological and/or psychological information about a patient. As with the FitBit, it may capture information about the patient's activity levels and may include monitoring of changes in heart rate. It may also include ambient monitoring devices such as fall-detectors in a home. Such information informs about changes over time as well as anomalies in the health of a patient relative to a healthy patient. Ongoing data may also include data from the patient's interaction with others, e.g., social media data.
4. As discussed in the embodiments above, the system also gathers ad hoc patient clinical data associated with clinician-patient interaction (in-office or remote). This was discussed in detail above, but for completeness is included here as part of the full complement of data captured about a patient for inclusion in the EHR system of the invention. The patient clinical data may include:
   a. Physiological data capture using electronic medical devices 110. As is discussed above, the different types of clinician-patient interaction data elements may be defined as resources to facilitate the mapping of the data to specific data locations in a data structure and to defined locations in a user portal)
   b. Verbal interactions (typically involving a microphone and which may include natural language processing (NLP) to transcribe the data. By semantically parsing of the transcribed data, it supports correlation of verbal data (once transcribed by NLP) with a dictionary of terms (medical terminology, and instructional words), in order to:
      i. Supplement electronic medical device data with comments by the physician or instructions to the patient (e.g., “Bend forward and breath in deeply”) to identify what part of the patient body was being monitored, in order to associate medical device data with specific data locations in a data structure and with defined location in a user portal, and
      ii. Support clinician supplementary notes by semantic parsing of audio or transcribed data, and comparing to a dictionary of medical terminology to identify phrases and words to be captured in Comment blocks.
   c. Video data (e.g., using one or more Wifi-enabled cameras) in order to:
      i. Provide additional physiological and psychological data about the patient, e.g., pallor of the patient's skin, and emotional state;
      ii. Complement data from medical devices e.g., to define the location on patient's body being monitored to assist in mapping medical device data to the correct data location, and
      iii. Provide a record of patient-clinician interaction for legal reasons, e.g., avoiding allegations of abuse or inappropriate behavior. In one embodiment, where a video record is to be retained for future use, the privacy of patients may be protected by generating avatars for the clinician and patient e.g. V-NECT software, and saving only the avatar interactions.
5. General symptomatic, genetic, biologic lab, and radiological or other medical imaging data. This may be incorporated into available patient data to inform the clinician and facilitate the rendering of a diagnosis. In addition to lab data, and data from third party EHR systems, the database of the system may be supplemented with medical journal information, and general research data relating symptomatic data to diagnoses.

In one implementation of the system, data from other EHR systems, lab data and image data is integrated into the EHR system of the invention by defining data elements that are to be assigned to specific data locations in the database and portal of the system, as resources. This complies with the terminology adopted by the FHIR (Fast Healthcare Interoperability Resources), which is a healthcare interoperability standard that is being mandated by CMS (Centers for Medicare and Medicaid Services) for implementation by Jul. 1, 2021. CMS is requiring health plans to provide interoperability and access to health data by enabling information exchange using the FHIR standard. FHIR is based on “Resources” which form the common building blocks for data exchanges by defining instance-level representations of healthcare elements. All resources have the following features in common:
A URL or identifier that identifies the resource,
Common metadata,
A human-readable XHTML summary,
A set of defined data elements (a different set for each type of resource), and
An extensibility framework to support variations.
   By way of example a patient is represented as a FHIR object in JSON as follows:

```
{
  “resourceType”: “Patient”,
  “id” : “23434”,
  “meta” : {
    “versionId” : “12”,
    “lastUpdated” : “2014-08-18T15:43:30Z”
  }
  “text”: {
    “status”: “generated”,
    “div”: “<!-- Snipped for Brevity -->”
  },
  “extension”: [
    {
      “url”: “http://example.org/consent#trials”,
      “valueCode”: “renal”
    }
  ],
  “identifier”: [
    {
      “use”: “usual”,
      “label”: “MRN”,
      “system”: “http://www.goodhealth.org/identifiers/mrn”,
      “value”: “123456”
    }
  ],
  “name”: [
    {
      “family”: [
        “Levin”
      ],
      “given”: [
        “Henry”
      ],
      “suffix”: [
        “The 7th”
      ]
    }
  ],
  “gender”: {
    “text”: “Male”
  },
  “birthDate”: “1932-09-24”,
  “active”: true
}
```

Each instance of a resource thus consists of:
resourceType (line 2 above),
id (line 3)—The id of this resource, which is always present when a resource is exchanged, except during the create operation,
meta (lines 4-7)—Usually Present and comprises Common use/context data
text (lines 8-11)—which is optional but helpful by providing a human readable representation for the resource,
extension (lines 12-17)—which is optional whenever an Extensions is required as defined by the extensibility framework of the FHIR specification,
data (lines 18-43)—comprises any data elements, which are different for each type of resource.

As indicated above, all resources may have a URL that identifies the resource and specifies where it was/can be accessed from.

Currently there are 145 defined resources types in the FHIR specification, which can be represented as either XML, JSON or RDF, thereby providing one method for implementing the present invention. The URLs thus facilitate mapping of data elements (resources) to locations in the database and portal by linking the URLs to field identifiers.

While the present invention has been described with respect to particular embodiments based on a predefined set of medical devices and a processor/server systems for analyzing the data, it will be appreciated that the invention can be implemented in different ways without departing from the scope of the invention of auto-populating an EHR with data using data from electronic medical devices and supplementing the device data with image and/or audio data, and preferably corroborating data from different medical devices. For instance, the corroboration may include time-relating data from the medical devices to data from one or more cameras and microphones that capture the interactions between a medical practitioner and a patient. The particular algorithm or use of an AI system to identify anomalies and corroborate data between devices, and to assign data to particular fields may also be implemented in different ways without departing from the scope of the invention.

The present invention thus allows a medical practitioner to automatically populate the fields in an EHR system in real time, rather than having to perform the data capture manually afterwards.

What is claimed is:

1. An EHR system comprising
a processor,
control memory connected to the processor that includes machine-readable code defining one or more algorithms for controlling the processor,
data storage,
at least one EHR user interface (UI),
a microphone,
a natural language processor (NLP),
and at least one medical device that captures medical data about a patient and is in communication with the EHR UI, wherein the data from each medical device is associated with one or more defined locations (also referred to herein as a data fields) in the EHR UI.

2. The system of claim 1, wherein the EHR UI comprises at least one portal defined by a web application (web app) accessible through a browser on a user device (using WiFi or a cell phone connection) or a native mobile application (App) that is downloaded to the user device

3. The system of claim 2, wherein the portal includes one or more of:
patient general information, including one or more of demographics, family history, and insurance;
pre-existing patient-specific clinical data;
continual or ongoing patient-specific data;
ad hoc clinical data associated with clinician-patient encounters, including one or more of: physiological data capture using electronic medical devices, verbal interactions, video data, and
genetic, and biologic lab data.

4. The system of claim 2, wherein the EHR UI includes multiple portals, including one or more of, a Clinician portal, a Patient portal, an Administrative portal, and a Payer portal.

5. The system of claim 4, wherein the Clinician portal defines data fields that include one or more of: procedures performed on a patient, patient medical condition information, medical findings, diagnosis, and prescriptions for the patient.

6. The system of claim 5, wherein the Clinician portal supports user input devices or a touch-sensitive screen, wherein the machine-readable code includes logic to allow a clinician to drag and drop data into user-defined fields in the Clinician portal.

7. The system of claim 4, wherein the Patient portal includes only select data to assist a patient with one or more of: scheduling, follow-up appointments, referrals, prescription fulfillment, and advisory health-care information.

8. The system of claim 4, wherein the Payer portal is limited to identifying the procedural steps performed and associated data in support of a reimbursement request.

9. The system of claim 3, wherein the system includes multiple modules, including one or more of: a referral module, a patient billing module and a reimbursement module, wherein the logic of the system associated with the reimbursement module, tracks reimbursements and calculates patient balance for processing by the patient billing module.

10. The system of claim 9, wherein the referral module includes logic for searching and identifying patient referral sources supported by a patient's insurance as defined by the patient general information.

11. The system of claim 1, wherein the machine-readable code is configured to control information from multiple data input sources, parse unstructured data, identify the fields in the EHR UI where the data is to be displayed, and keep a record of the data in the data storage.

12. The system of claim 1, further comprising at least one image capture device, for monitoring the activities of a medical practitioner in relation to the patient (also referred to herein as a procedure image capture device).

13. The system of claim 12, wherein image data from the at least one image capture device is parsed to identify body locations associated with activities performed on the patient in order to associate said activities with one or more data fields in the EHR UI.

14. The system of claim 12, wherein the machine-readable code includes an algorithm to corroborate data from one medical device with data from another medical device, the microphone or an image capture device, and flag physiological discrepancies.

15. An EHR system that comprises
a processor,
control memory connected to the processor, and configured with machine-readable
code to define an algorithms for controlling the processor,
data storage,
an EHR user interface (EHR UI),
at least one procedure image capture device, and
at least one medical device that captures medical data about a patient and is in communication with the EHR UI, wherein the algorithm includes logic for associating data from each medical device with one or more defined locations (data fields) in the EHR UI, and for identifying locations on a patient based on said at least one procedure image capture device.

16. A method of capturing patient information as part of a clinician-patient interaction (also referred to herein as a medical procedure or patient encounter), comprising
providing a user interface (UI) with multiple defined data entry fields,
providing one or more medical devices (also referred to herein as a medical instruments or medical sensors) that generate electronic data from physiological parameters of the patient,
capturing the electronic data from one or more of the medical devices and, for each said medical device, displaying its data on the UI in one or more data entry fields associate with said medical device,
and capturing at least one of: voice data, and video data to supplement the electronic data from the medical devices.

17. The method of claim 16, further comprising providing voice-transcription software to convert the voice data into text and entering the voice data into at least one of the data entry fields.

18. The method of claim 17, further comprising time-synchronizing the data from a medical device with the voice data.

19. The method of claim 18, further comprising parsing the voice data to assist in identifying the one or more data entry fields in the UI for said medical device data.

20. The method of claim 16, further comprising parsing the video data, time stamping the video data, and correlating the electronic data captured from at least one of the medical devices with the video data for the corresponding time frame.

21. The method of claim 20, wherein data from the voice-transcription software is associated with a data entry field based on one or more of key words and key phrases in the voice data, and the context of the key words and key phrases.

22. The method of claim 18, wherein the voice data is parsed to identify key words and key phrase, and in the case of a medical device being used during a corresponding time that the voice data is captured or within a defined time period of the voice data being captured, using said key words or key phrases of the voice data to provide additional information about the procedure that was performed using the medical device.

23. The method of claim 21, wherein image data captured by the procedure image capture device or by an image capture device associated with a medical device, is displayed in a common or associated field with data from the voice-transcription software.

24. The method of claim 16, wherein voice data, image data, and medical device data that is received, is compared to pre-stored data in order to identify physiological problems, and flagging said problems.
